(19) United States
(12) Patent Application Publication
(10) Pub. No.: US 2011/0245626 A1
(43) Pub. Date: Oct. 6, 2011

(54) System for human physical parameters examination

(76) Inventor: Chien-Liang Chen, Taipei (TW)

(21) Appl. No.: 12/010,660

(22) Filed: Jan. 28, 2008

(51) Int. Cl. A61B 5/00

(52) U.S. Cl. 600/301

(57) ABSTRACT

A system for detecting and recording a user's physical parameters, an interactive TV receiver connected to an information network system for receiving physical parameters data detected by the physical parameter scanner and transmitting the data to a rear end server and database through the information network system for enabling a remote user, remote hospital or remote pharmacy to trace patient's physical conditions and to provide the necessary medication services.

### BACKGROUND OF THE INVENTION

1. Field of the Invention

The present invention relates to interactive TV application technology and more particularly, to an interactive TV set-top box that provides a physical parameters examination function.

2. Description of the Related Art

The current interaction mode between a doctor and a patient is that the patient registers to see a doctor, and then goes to the hospital to receive the doctor's diagnosis subject to the registered schedule. To a patient living in a remote area, seeing a doctor needs to make a long and difficult journey. To an ordinary person, receiving a hospital diagnosis requires an appointment. One may have to change the routine schedule in order to see a doctor. Further, a chronic disease patient, for example, a hypertensive patient, diabetic patient or asthma patient may have to record specific physical parameters daily for diagnostic purpose. It is a difficult journey to a chronic patient to go to a hospital frequently for recording physical parameters data.

### SUMMARY OF THE INVENTION

The present invention has been accomplished under the circumstances in view. It is one object of the present invention to provide an interactive TV set-top box, which detects and records a user's physical parameters, for example, the daily physical parameters of a hypertensive patient, diabetic patient or asthma patient, for enabling a remote doctor or pharmacy to trace the patient's daily physical conditions and to provide the necessary medication services from a remote end.

To achieve this and other objects of the present invention, the interactive TV set-top box comprises a physical parameter scanner for detecting and recording a user's physical parameters, an interactive TV receiver connected to an information network system for receiving physical parameters data detected by the physical parameter scanner and transmitting the data to a rear end server and database through the information network system for enabling a remote user, remote hospital or remote pharmacy to trace patient's physical conditions and to provide the necessary medication services.

The invention interactive TV service content to join medical diagnosis and interactive TV services, enabling the user to provide physical parameters data to a remote doctor or pharmacist without making a long and difficult journey so that the remote doctor or pharmacist can trace the patient's daily physical conditions and provide the necessary medication services conveniently.

### BRIEF DESCRIPTION OF THE DRAWINGS

FIG. 1 is an installed view of the present invention.

FIG. 2 is a circuit block diagram of the present invention.

FIG. 3 is a schematic drawing showing an application example of the present invention.

### DETAILED DESCRIPTION OF THE PREFERRED EMBODIMENT

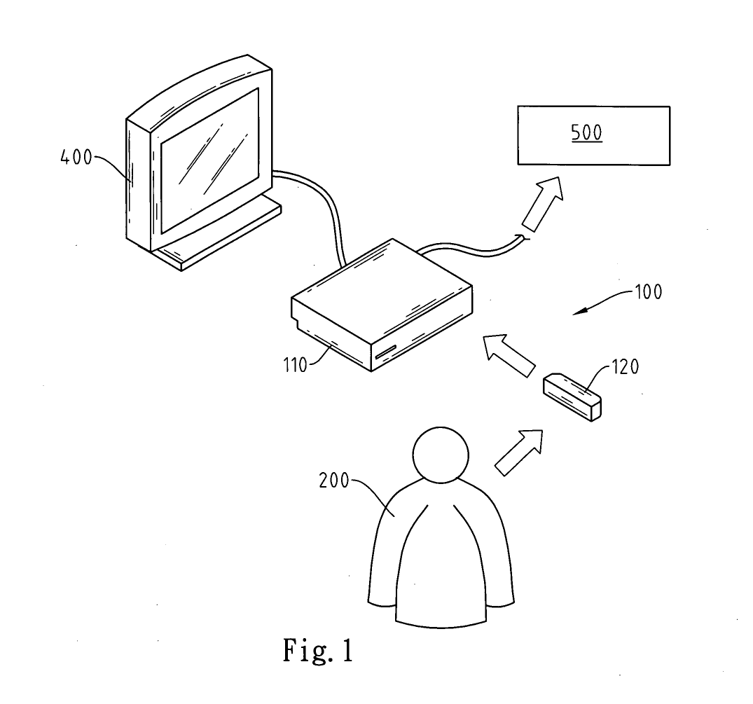

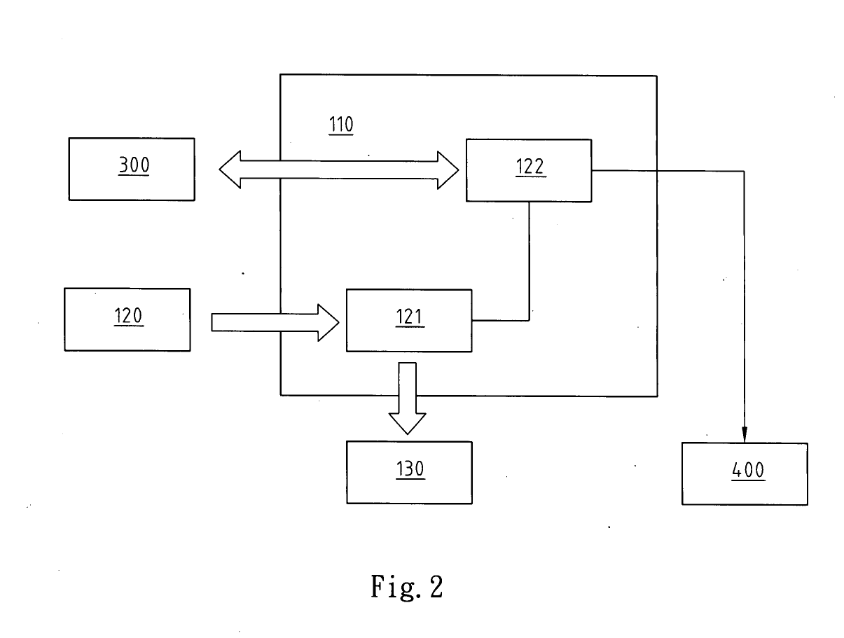

Referring to FIGS. 1 and 2, an interactive TV set-top box 100 is adapted to detect physical parameters of an user 200 and to transmit the detected physical parameters data to a rear end server 300 for use by a remote user.

The interactive TV set-top box 100 comprises an interactive TV receiver 110 and a physical parameter scanner 120.

The physical parameter scanner 120 is adapted for scanning and recording physical parameters of the user 200, such as body temperature, blood pressure, blood sugar concentration or glucose level, body weight, body fat percentage, and etc. The physical parameter scanner 120 may be made in any of a variety of forms subject to the items of physical parameters to be collected. It can be a clinical thermometer, blood pressure monitor, blood glucose scanner, weight scale, body fat scale, and etc., or any of their combinations. After the physical parameter scanner 120 scanned and recorded the related physical parameters of the user 200, the collected physical parameters data is transmitted to the interactive TV receiver 110 either by a cable or wireless apparatus.

The interactive TV receiver 110 comprises a control circuit 121 and a STB (set-top box) subsystem 122. The control circuit 121 is electrically connected with the STB subsystem 122 for controlling the operation of the STB subsystem 122. The STB subsystem 122 is connected to the rear end server 300 through an information network system, for example, the Internet or a CATV (cable television) cable area network, for data exchange with the rear end server 300. The STB subsystem 122 receives data from the rear end server 300 or the control circuit, and encodes the received data into a video signal for output to a monitor (TV or screen) 400 for display. Further, the control circuit 121 scans and controls the physical parameter scanner 120, and transmits data to the rear end server 300. Further, the STB subsystem 122 of the interactive TV receiver 110 receives interactive TV content from the rear end server 300 for display through the monitor 400.

The physical parameter scanner 120 is connected to the control circuit 121 (by a cable or wireless apparatus), so that the control circuit 121 receives physical parameters data from the physical parameter scanner 120 and transmits the data to the STB subsystem 122 that in turns transmits the data through the information network system to the rear end server 300 for further processing and use.

The interactive TV set-top box 100 further comprises an input interface 130. The input interface 130 can be a remote controller having direction buttons and a keypad, a discrete keyboard, a mouse, or a joystick. The input interface 130 is connected to the control circuit 121 of the interactive TV receiver 110 for allowing the user to input a description of the physical status. Alternatively, a menu interface that provides a list of physical status description items can be displayed on the monitor 400 so that the user can selectively click on the physical status description items for transmission with the detected physical parameters data to the rear end server 300. The menu interface can be built in the control circuit 121 or STB subsystem 122, or fetched from a remote source through the information network system.

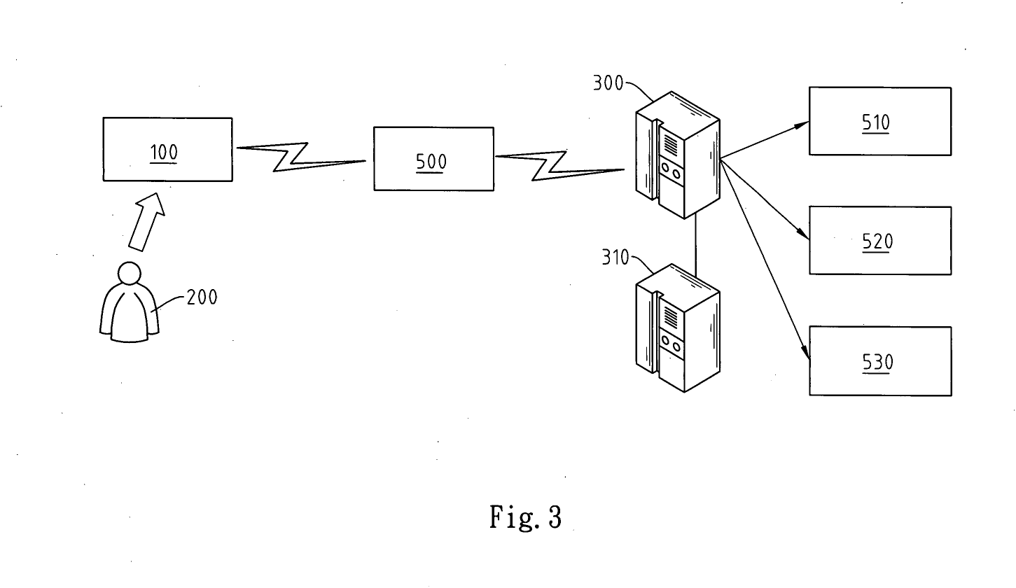

Referring to FIG. 3, after obtained the user's physical parameters, the interactive TV set-top box 100 reads the data and displays the data on the monitor 400 via the interactive TV receiver 110 for user's reference. The user can input personal ID (identification) number or code before or after examination. Upon receipt of the user user's ID number or code, the interactive TV receiver 110 transmits the data to the rear end server 300 via the information network system 500.

When receipt of the user's physical parameter and ID data, the rear end server 300 stores the data in a database 310. The aforesaid menu interface that provides a list of physical status description items is stored in the rear end server 300 or the database 310. The menu interface changes the content of its list of physical status description items subject to the inputted personal ID number or code inputted through the interactive TV set-top box 100, and is provided to the interactive TV set-top box 100.

The individual physical parameters data stored in the database 310 includes the latest record and history record, and is transmittable to a remote end, such as hospital 510, pharmacy 520, remote user (doctor or pharmacist) 530 for analysis by a professional person to trace the user's health condition. The related instructions can be fed back to the interactive TV set-top box 100 through the rear end server 300 for user's reference. The pharmacy 520 can also prepare drug for long-term supply subject to the conditions of the user'who is taking long-term medications.

The invention can be used to record the user's physical condition data in detail, and to store the data in the remote-end database 310 for review and tracing when desired. The doctor or pharmacist can fetch the related data from the remote-end database 310 to review the user's physical conditions. By means using the interactive TV set-top box 100 with a monitor or TV, medical diagnosis and interactive TV services are joined, and therefore the user can obtain medication service conveniently without making a long and difficult journey or changing one's routine schedule to see a doctor

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A system for human physical parameters examination, comprising:
a physical parameter scanner adapted to detect and record a user's physical parameters; and
an interactive TV receiver connected to an information network system and adapted to receive physical parameters data detected by said physical parameter scanner and to transmit the data to a remote end through said information network system for processing.

2. The system for human physical parameters examination as claimed in claim 1, wherein said interactive TV receiver comprises:
a set-top box subsystem connected to said information network system for data exchange and for displaying data on a monitor; and
a control circuit electrically connected to said set-top box subsystem for controlling operation of said set-top box subsystem.

3. The system for human physical parameters examination as claimed in claim 2, wherein said physical parameter scanner is connected to said control circuit by a cable.

4. The system for human physical parameters examination as claimed in claim 2, wherein said physical parameter scanner is connected to said control circuit wirelessly.

5. The system for human physical parameters examination as claimed in claim 2, wherein said control circuit scans and controls said physical parameter scanner, and transmits data obtained from said physical parameter scanner to a remote end through said information network system.

6. The system for human physical parameters examination as claimed in claim 2, wherein said set-top box subsystem is connected to a rear end server through said information network system for data exchange.

7. The system for human physical parameters examination as claimed in claim 6, further comprising a database electrically connected to said rear end server for storing users' physical parameters data subject to the control of said rear end server.

8. The system for human physical parameters examination as claimed in claim 6, wherein said rear end server is controllable to transmit users' physical parameters data to a hospital, a pharmacy, or a remote user.

9. The system for human physical parameters examination as claimed in claim 1, wherein said physical parameters include the user's body temperature, blood pressure, blood sugar concentration, body weight, and/or body fat percentage.

10. The system for human physical parameters examination as claimed in claim 1, wherein said physical parameters canner is a clinical thermometer, blood pressure monitor, blood glucose scanner, weight scale, body fat scale, or their combination.

11. The system for human physical parameters examination as claimed in claim 1, further comprising an input interface connected to said interactive TV receiver for allowing a user to input a description of physical status.

12. The system for human physical parameters examination as claimed in claim 1, further comprising an input interface connected to said interactive TV receiver to provide a menu interface for allowing a user to choose physical status description items.
